(19) United States
(12) Patent Application Publication
(10) Pub. No.: US 2018/0055686 A1
(43) Pub. Date: Mar. 1, 2018

(54) APPARATUS FOR APPLYING COLD THERAPY TO A JOINT OF A PERSON OR AN ANIMAL

(71) Applicant: Mariti Antonio MUNOZ, Brownsville, TX (US)

(72) Inventor: Mariti Antonio MUNOZ, Brownsville, TX (US)

(21) Appl. No.: 15/246,120

(22) Filed: Aug. 24, 2016

(51) Int. Cl. A61F 7/02; A61F 7/10

(52) U.S. Cl. CPC A61F 7/02; A61F 2007/0044; A61F 7/10

(57) ABSTRACT

An assembly for cold therapy to a joint of a person or an animal has a panel formed of a foam material, and a plurality of ice packs positioned within the foam material of the panel in a location between an inner side and an outer side of the panel. The panel is adapted to wrap around at least a portion of the joint. The assembly further includes a wrap extending across the outer side of the panel. This wrap has a length suitable for wrapping around the joint. The plurality of ice packs are arranged in rows and columns within the panel. The foam material is a polyurethane open cell foam material. The wrap is formed of an elastomeric material. The wrap has a fastener thereon that is adapted to affix one end of the wrap to an outer surface of the wrap.

### CROSS-REFERENCE TO RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### INCORPORATION-BY-REFERENCE OF MATERIALS SUBMITTED ON A COMPACT DISC

Not applicable.

### BACKGROUND OF THE INVENTION

### 1. Field of the Invention

The present invention relates to cold therapy. More particularly, the present invention relates to the application of ice packs onto a human body. In particular, the present invention relates to the use of ice packs in a wrap that is applied around a joint of a person or an animal.

### 2. Description of Related Art Including Information Disclosed Under 37 CFR 1.97 and 37 CFR 1.98

Cold compression therapy combines the principles of rest, ice, compression and elevation to reduce pain and swelling from a sports or activity injury to soft tissues. It is also recommended by orthopedic surgeons following surgery. The therapy is especially useful for sprains, strains, pulled muscles and pulled ligaments. Cold compression is a combination of cryotherapy and static compression, commonly used for the treatment of pain and inflammation after acute injury or surgical procedures. The use of ice or cold in cryotherapy has been one of the most common treatments in orthopedic medicine. The primary use in reason for use in cryotherapy in acute injury management is to lower the temperature of the injured tissue, which reduces the tissue's metabolic rate and helps the tissue to survive the period following the injury. It is well understood that metabolic rate decreases by application of cryotherapy.

Static compression is often used in conjunction with cryotherapy for the care of acute injuries. Today, the primary reason for using compression is to increase external pressure on the tissue to prevent edema formation (i.e. swelling). This occurs by hindering fluid loss from the vessels in the injured area, making it more difficult for fluids to accumulate. Ice with compression is significantly colder than ice alone due to the improved skin contact and increased tissue density caused by extended static compression. Tissue reaches its lowest temperature faster and the tissue maintains its cool even after the treatment ends.

Continuous cold therapy devices circulate ice water through pads. These are currently the subject of class action lawsuits for skin and tissue damage caused by excessive cooling/icing time and lack of temperature control. Reported injuries range from frostbite to severe tissue damage that can result in amputations. Studies of shown that the body activates a hunting response after only ten minutes of cryotherapy at temperatures less than 49° F. The hunting response is a cycle of vasoconstriction (i.e. decreased blood flow), then vasodilation (i.e. increased blood flow) that increases the delivery of oxygen and nutrient rich blood to the tissue. Increased blood flow can slow cell death, limit tissue damage and aid in the removal of cellular debris and waste products. Under normal circumstances, the hunting response would be essential to tissue health that only serves to increase pain, inflammation and cell death as excess blood is forced into the area.

Cold compression wraps, using either re-freezable ice or a gel, are much safer products. These products do not exceed the cooling/icing time recommended by the established medical community. Many of the ice wraps available use adjustable elastic straps to aid in compression over the injured areas. Many advanced single-use wraps have guidelines to indicate how the bandage should be applied in order to achieve optimum compression required for an acute injury. Most ice wraps that use ice have a built-in protective layer so that the ice is not applied directly to the skin.

Equine lameness has many causes, of which laminitis is among the most serious. Laminitis, often called founder, is disease that affects nearly 15% of the horse population and results in debilitation of approximately 75% of these horses. Despite recent intensive investigation, no cure currently exist, but early detection and prompt treatment can prevent the need for euthanasia. Laminitis is a fast-acting disease that attacks and breaks down the connective tissue, the laminae, between the horse's wall and coffin bone. The disease occurs in three successive stages: the developmental, the acute, and the chronic. The development stage of laminitis occurs between initiation of the disease and the appearance of acute lameness and lasts a maximum of 72 hours. The acute stage can last between 4 and 60 hours. This means that many horses may already be in the chronic stage of the disease before they receive treatment. Therefore, until an actual cure is found, and feasible method for preventing and treating the disease is needed.

While a cure for laminitis may be in the future, recent research on the use of cold therapy is a potential prophylactic offers immediate hope for preventing the disease. Studies have found that continuous cryotherapy is well tolerated and result in market cooling of the treated foot.

Additionally, it is desirable to apply a cold compressive force to the legs of a horse in other treatment protocols. There exist numerous devices of a myriad of constructions that apply compressive force alone or in combination with cold or heat therapy. However, there remains a need for a leg wrap of an improved construction that is configured to wrap around and conform to the complex anatomy of the front and rear legs of a horse and to apply cold therapy to the leg.

In the past, various patterns of issued relating to equine cold therapy. For example, U.S. Pat. No. 3,717,145, issued on Feb. 20, 1973 to Berndt et al., teaches a cold bandage for application to various portions of the body of an animal. The bandage includes an outer enclosure containing at least one bag of a volatile refrigerant with an opening in the bag to enhance vaporization of the refrigerant. The outer enclosure is in the form of an envelope that is sealed gas tight with at least one bag opening into the interior of the envelope. The bandage becomes inflated by virtue of the refrigerant volatilizing and intimately conforms to the portion of the body to which the bandage is applied. This provides both reduce temperature and pressure.

U.S. Pat. No. 3,822,705, issued on Jul. 9, 1974 to R. Pilotte, teaches a refrigerant wrap for an animal's limb. The device contains a refrigerant therein. In particular, there are a plurality of refrigerant cells held side-by-side into a series of adjoining pockets interspaced by fold lines for simple manufacturing and to equally wrap around the limb or leg for a relatively uniform cooling treatment. The refrigerant wrap includes attached bands of hook-and-loop material. The refrigerant wrap also includes a wire mesh that is at least partially shapable according to the limb upon which the wrap is placed.

U.S. Pat. No. 3,871,381, issued on Mar. 18, 1975 to D. Roslonski, describes a cold compress device for treating injuries in mammals. The cold compress device includes a flexible inflatable compress adapted to be placed adjacent an area to be treated and a source of compressed refrigerant connected to the compress. A channel is formed in a portion of the compress wall which must be traversed by incoming refrigerant before it enters the interior of the inflatable compress. The device also includes means interposed between the source of the refrigerant and the compress for controlling the rate of flow of refrigerant from the source into the compress. The compressed refrigerant expands in the compress to simultaneously cool and inflate the compress.

U.S. Pat. No. 5,152,285, issued Oct. 6, 1992 to B. D. Gnegy, provides a boot system for selectively applying heat or cold to the lower portion of the leg of a horse. There is a U-shaped inner boot portion positioned over the portion of the leg at a front portion thereof. The inner boot portion has a leather front panel with a pair of side panels extending from the front panel. The inner boot portion is lined with the fabric portion of a touch fastener material and has a hook portion on the outer surface of the side panels. There is also a leather-covered U-shaped outer boot portion positioned over the back of the leg with a pair of side panels overlapping the pair of side panels of the inner boot portion and lined with the fabric portion of the touch fastener material being releasable engagement with the hook portion on the outer surface of the pair of panels. A plurality of pockets each have a hook portion of the touch fastener material on a surface thereof and releasably attached to the fabric portion of the touch fastener material at a point where heat or cold is to be applied to the lower portion of the leg. A plurality of gel-filled hot/cold packs are disposed within the respective ones of the pockets.

U.S. Pat. No. 8,166,734, issued on May 1, 2012 to M. L. Ruetnik, discloses an equine cold therapy apparatus and method. The apparatus is a flexible boot with an orthotic pad that has various features to allow for circulating coolant so that the bottom of the hoof area of an equine can be cooled. The pad is also designed to allow cooling under vacuum conditions. The apparatus provides elements to facilitate cooling the lower leg of the equine in a way designed to allow coolant circulated through the boot pad to be also circulated through the leg cooling elements while maintaining a controlled temperature against the leg surface.

U.S. Patent Application Publication No. 2010/0050959, published a Mar. 4, 2010 to C. H. McPeak-Young, teaches front and rear leg wraps for applying cold or heat compression therapy to the legs of a horse. A series of horizontally-extending pockets extend along a length of the wrap. The pockets are filled with refrigerant bags containing a quantity of refrigerant material. The quality of refrigerant material contained in the refrigerant bags can be applied to control the amount of cold or heat therapy to be applied. The front and rear leg wraps include gussets to permit the wraps to closely conform to the complex anatomy of the horse's leg.

It is object of the present invention to provide a device for cold therapy that both provides compression and cooling relief to a limb or a joint of a human or an animal.

It is another object of the present invention to provide a device for cold therapy which minimizes possible frostbite or damage to the skin upon which the device is applied.

It is another object of the present invention to provide a device for cold therapy which can conform to the configuration of the particular joint of the human or animal.

It is still another object of the present invention provide a device for cold therapy which can be quickly secured to the treated area.

It is still another object of the present invention to provide a device for cold therapy which is easy to use, easy to manufacture and relatively inexpensive.

It is still further object the present invention to provide a device for cold therapy that effectively treats laminitis in the leg and ankle joint of a horse.

These and other objects and advantages of the present invention will become apparent from a reading of the attached specification and appended claims.

### BRIEF SUMMARY OF THE INVENTION

The present invention is a device for cold therapy that comprises a panel formed of a foam material and a plurality of ice packs positioned within the foam material of the panel in a location between an inner side and an outer side of the panel. The plurality of ice packs are arranged in rows and columns within the panel. The foam material is a polyurethane open cell foam material.

A wrap can extend around the outer side of the panel. This wrap is of an elastomeric material, such as neoprene. The wrap has a fastener thereon. The fastener is in the nature of hook-and-loop material and is adapted to affix one end of the wrap onto an outer surface of the wrap.

The present invention is also an assembly for cold therapy to the joint of a person or an animal. This assembly includes a panel formed of a foam material which is adapted to wrap around at least a portion of the joint, and a plurality of ice packs positioned within the material in a location between the inner side and the outer side of the panel. The wrap has a length suitable for wrapping entirely around the joint. In particular, in this assembly, the joint is an ankle of a horse.

This foregoing Section is intended to describe, with particularity, the preferred embodiments of the present invention. It is understood that modifications to these preferred embodiments can be made within the scope of the present invention. As such, this Section should not to be construed, in any way, as limiting of the broad scope of the present invention. The present invention should only be limited by the following claims and their legal equivalents.

### BRIEF DESCRIPTION OF THE SEVERAL VIEWS OF THE DRAWINGS

FIG. 1 is an inner perspective view of the cold therapy device of the present invention.

FIG. 2 is a cross-sectional view of an ice pack as used within the foam material of the panel of the cold therapy device of the present invention.

FIG. 3 is a side elevational view showing the application of a protective boot onto the ankle of a horse.

### DETAILED DESCRIPTION OF THE INVENTION

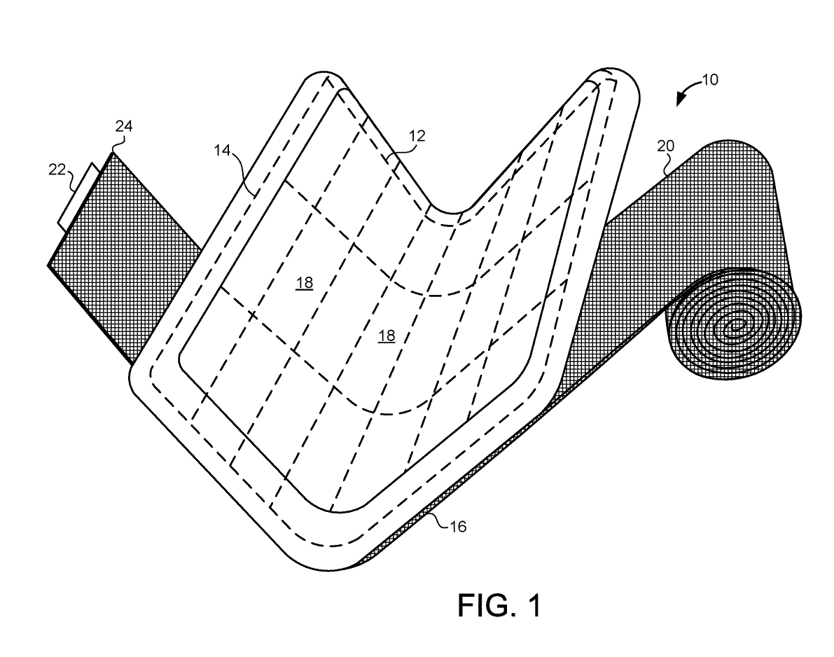

Referring to FIG. 1, there is shown the cold therapy device 10 in accordance with the present invention. The cold therapy device 10 includes a panel 12 formed of a foam material. The panel 12 has an inner side 14 and an outer side 16. A plurality of ice packs 18 are positioned within the foam material of the panel 12 in a location between the inner side 14 and the outer side 16 of the panel 12. As can be seen in FIG. 1, the plurality of ice packs 18 are arranged in rows and columns within the panel 12. The foam material is, preferably, a polyurethane open cell foam material.

A wrap 20 extends across the outer side 16 of the panel 12. This wrap 20 is of an elastomeric material. In particular, the elastomeric material of the wrap 20 is neoprene. The wrap 20 has a fastener 22 thereon. This fastener is in the nature of hook-and-loop material that is adapted to affix one end 24 of the wrap 20 onto an outer surface of the wrap when the wrap is placed around a joint.

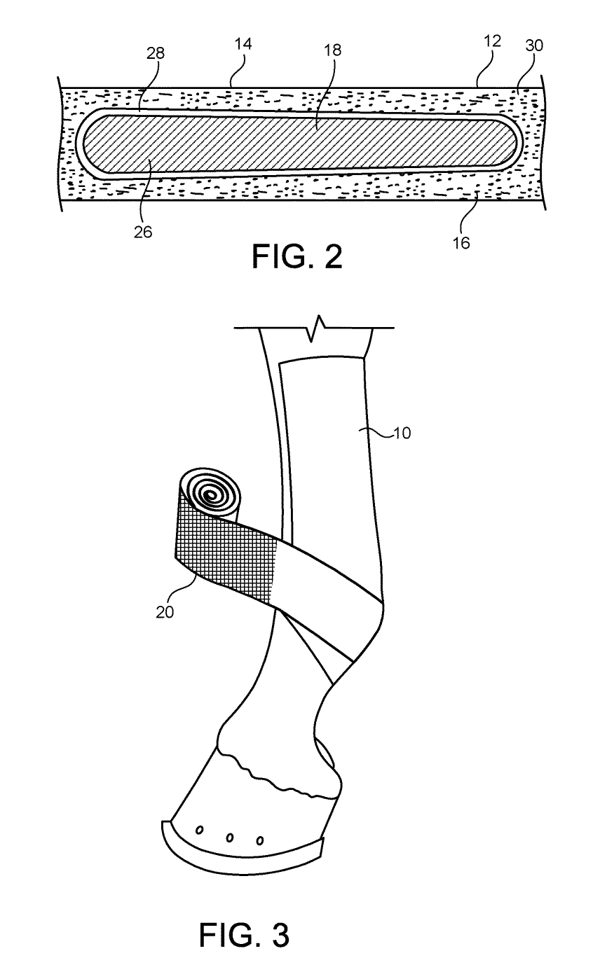

Importantly, with reference to FIG. 2, the ice pack 18 is retained in an interior of the panel 12 between the inner side 14 and the outer side 16. The ice pack 18 is a conventional ice pack in which a liquid or a gel 26 is retained within an envelope 28. The ice pack 18 is a portable plastic sack filled with water or a refrigerant gel or liquid. For use, the contents are frozen in a freezer. In particular, the present invention, the panel 12 can be entirely placed into the freezer so that the liquid 26 becomes frozen or cooled. Both ice and other non-toxic refrigerants can absorb a considerable amount of heat before they warm to above freezing due to the lack of latent heat of fusion of water. The ice packs 18 are commonly used to keep food cold in portable coolers or as a cold compress to alleviate the pain of minor injuries, or in insulated shipping containers to keep product cool during transport. The envelope 28 is formed of a flexible material and is strong enough to retain the refrigerant 26 within the interior thereof.

In FIG. 2, the panel 12 is formed of the foam material 30. The foam material is a polyurethane open cell foam. During the molding of the ice pack 18 within the foam material 30, the ice pack 18 is retained in position so that it is not exposed on either the inner side 14 or the outer side 16 of the panel 12. As such, the foam material 30 will actually be interposed between the ice pack 18 and the inner side 14 and the outer side 16. This provides an amount of insulation so as to prevent direct contact between the ice pack and the skin of the user. As such, frostbite and other damaging contact with skin is avoided.

The foam material 30 is a pillow-soft foam. As such, it is very comfortable when applied to the skin. Although covers or other materials could be applied to the inner surface 14 and the outer surface 16 of the panel 12, it is believed that the softness of the foam material 30 will avoid the need to include these other materials. Additionally, this foam material 30 is sufficiently flexible so as to easily conform to the surfaces of the joint of a human or animal. Since the ice packs 18 are also very flexible, the cold therapy device 10 of the present invention can be easily molded around the joints so as to consistently and evenly apply cold therapy. The cold therapy device 10 is easy to reuse. Once treatment is complete, the panel 12, including the ice packs 18, can be simply placed in a refrigerator so as to cool the ice packs once again. The wrap 20 allows the panel 12 to be quickly and easily applied and removed.

FIG. 3 shows that the wrap 20 is be unrolled and wrapped around the cold therapy device 10. As such, the wrap 20 will establish a firm and compressive relationship between the inner surface of the wrap 20 and the outer surface of the cold therapy device 10. Once the wrap 20 is entirely wrapped around the cold therapy device 10, the fastener 22 can be secured to the outer surface of the wrap 20 so as to fix the wrap in position.

The present invention allows compressive cold therapy to be applied to the ankle of the horse while avoiding any potential injury as a result of the cold contact between the cold therapy device 10 and the skin of the horse. A cooling effect is more closely transmitted to these areas of concern. As such, the unique application of the cold therapy device 10 achieves unique effects in the application of cold therapy to the ankle of the horse. Additionally, the continued application of cold therapy to the ankle of the horse can serve to avoid the dangerous effects of laminitis. After the cold therapy is completed, the fastener is unfastened from the surface of the wrap and the wrap is unrolled from the cold therapy device 10. The cold therapy device 10 can then removed from the ankle of the horse.

The foregoing disclosure and description of the invention is illustrative and explanatory thereof. Various changes in the details of the illustrated construction can be made within the scope of the appended claims without departing from the true spirit of the invention. The present invention should only be limited by the following claims and their legal equivalents.

I claim:

1. A device for cold therapy, the device comprising:
a panel formed of a foam material, said panel having an inner side and an outer side; and
a plurality of ice packs positioned within said foam material of said panel in a location between said inner side and said outer side of said panel.

2. The device claim 1, said plurality of ice packs arranged in rows and columns within said panel.

3. The device of claim 1, said foam material be in a polyurethane open cell foam material.

4. The device of claim 1, further comprising:
a wrap extending around said outer side of said panel.

5. The device of claim 4, said wrap being of an elastomeric material.

6. The device of claim 5, said elastomeric material being neoprene.

7. The divisive claim 4, said wrap having a fastener thereon, said fastener adapted to affix one end of said wrap to a surface of the wrap.

8. An assembly for cold therapy to a joint of a person or an animal, the assembly comprising:
a panel formed of a foam material, said panel having an inner side and an outer side, said panel adapted to wrap around at least a portion of the joint; and
a plurality of ice packs positioned within said foam material of said panel in a location between said inner side and said outer side of said panel.

9. The assembly of claim 8, said plurality of ice packs arranged in rows and columns within said panel.

10. The assembly of claim 8, further comprising:
a wrap extending across said outer side of said panel, said wrap having a length suitable for wrapping entirely around the joint.

11. The assembly of claim 10, said wrap being of an elastomeric material.

12. The assembly of claim 10, said wrap having a fastener thereon, said fastener adapted to affix one end of said wrap to a surface of the wrap when said wrap is wrapped around the joint.

13. An apparatus for applying cold therapy to an ankle of the horse, the apparatus comprising:
a panel formed of a foam material, said panel having an inner side and an outer side, said inner side of said panel facing an inner surface of the ankle of the horse; and
a plurality of ice packs positioned within said foam material of said panel in a location between said inner side and said outer side of said panel.

14. The apparatus of claim 13, further comprising:
a wrap extending across said outer side of said panel, said wrap extending around said outer side of said panel.

15. The apparatus of claim 14, said wrap being of a neoprene material, said foam being a polyurethane open cell foam material.
